(19) United States
(12) Patent Application Publication
(10) Pub. No.: US 2026/0224342 A1
(43) Pub. Date: Aug. 6, 2026

(54) DENTAL UNIT WATERLINE SYSTEM, KIT AND METHOD OF USE

(71) Applicant: Parkell, Inc., Edgewood, NY (US)

(72) Inventors: David Mott, West Bay Shore, NY (US); Michael Karter, Dix Hills, NY (US)

(21) Appl. No.: 19/531,906

(22) Filed: Feb. 6, 2026

Related U.S. Application Data
(60) Provisional application No. 63/755,136, filed on Feb. 6, 2025.
(60) Provisional application No. 63/903,351, filed on Oct. 22, 2025.

(51) Int. Cl. A61C 19/00; A61L 2/18; A61L 101/36; A61L 101/44; A61L 103/15; B01D 69/02; B01D 71/68; B08B 9/032; C02F 1/00; C02F 1/44; C02F 1/50

(52) U.S. Cl. CPC A61C 19/002; A61L 2/18; B01D 69/02; B01D 71/68; B08B 9/0321; C02F 1/001; C02F 1/444; C02F 1/50; A61L 2101/36; A61L 2101/44; A61L 2103/15; B01D 2325/36; B08B 2209/032; C02F 2303/04

(57) ABSTRACT

A dental system or kit has components including a water filter, a dental unit, and a handpiece connected with connectors and waterlines having quick disconnect fittings. The components define a water flow path from a water source to the handpiece and can be easily and quickly disconnected at any point along the flow path to access the flow path. A syringe including a disinfecting and/or sterilizing agent is provided to inject the disinfecting and/or sterilizing agent into the flow path to sterilize and/or disinfect the water flow path downstream of the injection site. A self-contained reservoir assembly in communication with a dental unit includes a reservoir assembly and a water filter including a chemical free-filter member.

### CROSS-REFERENCE TO RELATED APPLICATIONS

This application claims benefit of and priority to U.S. Provisional Application No. 63/755,136, filed Feb. 6, 2025, and U.S. Provisional Application No. 63/903,351, filed Oct. 22, 2025, which are incorporated by reference in their entireties.

### TECHNICAL FIELD

The disclosure relates to a system, a kit and a method for disinfecting and/or sterilizing the water lines of dental equipment, as well as for preventing and/or minimizing bacterial contamination of dental unit waterlines.

### BACKGROUND

Dental chairs and treatment equipment, i.e., dental units, are supposed to deliver only “drinking” quality water to patients and staff in the form of liquid (which enters the patients' mouths) as well as in the form of aerosols which are produced by the dental units and enter the dental office air around patients and staff. “Drinking water” is defined by the Environmental Protection Agency (EPA) as water than contains less than 500 colony-forming units (CFU) per milliliter (CFU/mL) of bacteria. However, as described herein, as high as 70% of dental practices in the United States fail to properly maintain waterlines within dental units and fail to ensure delivery of only “drinking” quality water to patients and staff, for example, because they fail to adhere to warnings about biofilm infiltration and colonization within the internal and external waterlines of dental units. This results in dental unit waterlines that can contain bacterial and biofilm loads which far exceed the EPA standard for safety and, therefore, in significant risks to both patients and dental staff, such as in the form of serious respiratory or other bacterial infections (e.g., legionella). As described herein, this has in fact resulted in several bacterial outbreaks, particularly amongst children, geriatric patients, and immuno-compromised patients. For these reasons, there remains a significant and long felt need in the dental industry for improved systems and methods for preventing bacterial and other microbial agents from entering dental unit waterlines (DUWLs) as well as improved systems and methods for decontaminating DUWLs which already contain bacteria and biofilm. Such improved systems and methods are presented herein.

Non-limiting examples of dental units which include waterlines and which are at risk of bacterial contamination include dental chairs and associated waterlines, dental water bottle systems and/or self-contained systems (e.g., reservoir systems), ultrasonic scalers, air-water syringes, high-speed handpieces, low-speed handpieces, air polishing units, handpieces, or similar dental units—each of which may include one or more waterlines for delivering treatment (or procedural) water from a source of municipal water or with one or more liquid containers (e.g., bottles, for example, which are filled by dental practitioners such as from tap or other water).

The dental unit waterlines (DUWLs) are integral components of dental units. They are important for several reasons, such as but not limited to irrigation, cooling, flushing, and purging before, during, or after dental procedures (such as, for example, to prevent cross-infect between patients). DUWLs can be constructed of polymers (e.g., polyurethane, polyvinyl chloride, or the like) or silicone rubber or the like.

According to the US Food and Drug Administration (FDA), dental unit waterlines, including those connected to municipal water sources or reservoir systems (e.g., closed-bottle systems), typically cannot be sterilized; however, they should be routinely tested, cleaned, and disinfected. See, https://www.fda.gov/medical-devices/dental-devices/dental-unit-waterlines.

Although there are standard methods for determining bacterial concentrations in water, there is no universal agreement on the acceptable concentration of organisms in drinking water. See, National Research Council (US) Safe Drinking Water Committee. Drinking Water and Health: Volume 1. Washington (DC): National Academies Press (US); 1977. III, Microbiology of Drinking Water. Available from: https://www.ncbi.nlm.nih.gov/books/NBK234164. The most common allowable bacterial numbers used by health departments, water-supply agencies, and local jurisdictions vary from 100/ml to 500/ml of colony-forming units. Id. The U.S. Centers for Disease Control and Prevention (CDC) recommends that dental unit water used in nonsurgical procedures measure less than or equal to 500 colony forming units of heterotrophic bacteria per milliliter (≤500 CFU/mL) of water, the standard set for drinking water by the Environmental Protection Agency (EPA). Moreover, certain U.S. States have enacted versions of the CDC standard into law, such as Washington and Georgia.

Even at the connection point of the drinking water supply and the dental equipment or units, there is often found a bacteria count that is far above 100 or 500 colony-forming units per milliliter and there are often times detected problematic bacteria such as “Pseudomonas aeruginosa” or “Legionella”. In addition, dental units can have particular features in contrast to the drinking water system, which promote bacteria growth and causes a high bacteria count.

A dental unit can comprise any number of dental unit waterlines (e.g., one or more dental unit waterlines) in fluid communication from a water source to one or more handpieces or other dental units (e.g., treatment devices or handpieces). Dental units are used by dental professionals to perform procedures in or proximate to patients' mouths. For this reason, dental units with contaminated waterlines pose a serious public health risk. Infection of patients or staff can occur from contaminated dental units in several different ways, including but not limited to: (i) transfer of bacteria or pathogens in treatment water to the oral or nasal cavity of the patient (e.g., when splatter occurs during dental procedures), and (ii) transfer of bacteria or pathogens within aerosols (and thus the dental office air) which are commonly generated during dental procedures, and which can lead to respiratory infections in patients and/or staff. For this reason, improved systems and methods for dental unit waterline maintenance are a strongly felt problem in the dental industry and is increasingly being viewed as a public health crisis in dentistry. See, e.g., Spagnolo et al., Microbial Contamination of Dental Unit Waterlines and Potential Risk of Infection: A Narrative Review. Pathogens. 2020 Aug. 13; 9(8):651.

Bacterial and/or biofilm contamination of dental unit waterlines (DUWLs) can occur in a variety of ways, such as from the source water side and/or from the patient treatment side. A very common source for bacteria in DUWL's is source water. In particular, for example, bacteria can enter DUWLs directly from source water, such as from municipal, bottled, or even water that is chemically treated with commercially-available straws or tablets (which may contain bacteria due to the fact that, e.g., the straws and tablets release only low concentrations of antimicrobial agent, e.g., iodine or silver—often insufficient concentrations to kill all bacteria present in the source water—and are highly sensitive to the chemical composition of the source water which can cause the systems to fail and permit contaminated water to enter dental units and associated waterlines)).

The microorganisms (e.g., bacteria and pathogens) that inhabit source water (e.g., from a municipal water source or from a reservoir system, e.g., closed-bottle system) colonize DUWLs and adhere to the inner walls of the DUWLs or other components of dental units. Once in the dental unit or DUWL, bacteria or other microorganisms reside, replicate, and multiply in number, ultimately forming thick biofilms that are extremely difficult to remove and which ultimately shed into treatment water and air (in the form of aerosols). See, Spagnolo et al., supra.

In this regard, it is important to note that contaminated dental treatment water (such as dental treatment water having greater than 100 CFU/mL or even greater than 500 CFU/mL of bacteria) is unsafe to patients and staff not only in terms of water entering the oral cavity during dental procedures, but also raises significant health and infection concerns because the treatment water is, by design, aerosolized by certain dental or treatment units (e.g., ultrasonic scalers and high-speed handpieces) and therefore presents significant infection risks in dental office air and has been associated with serious respiratory infections of staff and patients.

Alternatively, or in addition, bacterial contamination may occur when the dentist or hygienist switches off the spray of water and air at the end of a particular operation. In this situation, when the flow of air and water stops, ideally, no water drips from the handpiece. In instances, this is because the minor residual pressure in the conduits tending to push the water out is counteracted by a slight pressure in the opposite direction activated to prevent the water from dripping out. Unfortunately, saliva can be drawn back into the waterline in this scenario.

A handpiece designed to operate in this way, although it does prevent water from dripping out, may allow bacteria to be sucked into the handpiece itself from the patient's mouth, causing particles of tissue from the patient's mouth to be introduced within the handpiece. In other situations, suck-back of patients' saliva into the line can occur due to the lack of adequate valve function. As a result, any germs and viruses present in the patient's mouth may enter the water conduit through the handpiece and from there may contaminate the entire water line of the dental unit.

The presence of bacteria and/or micro-organisms in dental unit waterlines and/or associated liquid reservoirs or containers (e.g., closed-bottle systems)—and, thus, in dental treatment water and air—can present significant health hazards in the form of serious respiratory or other infections. This is especially dangerous for high-risk patients, such as children, geriatric patients, and/or immunocompromised patients. Moreover, risks associated with contaminated (e.g., high bacteria load) treatment water presents significant health hazards during dental treatment procedures where the oral mucosa is at risk of being breathed or damaged, such as during routine ultrasonic scalers. Moreover, during the use of handpieces, particularly high-speed rotating instruments, a spray is thrown up in the form of aerosols or spatters containing biological material (saliva, blood and dental plaque) and micro-organisms. This means that the health of both dental staff and patients could be at risk of infection. Spagnolo et al., supra.

Some examples of serious bacterial infections that have occurred include: (i) in 2016, over 70 children (ages 4-8) were infected with Mycobacterium abscesses which were traced to dental water in an Orange County, California dental practice. The impacted children were hospitalized for an average of more than a week and some suffered permanent tooth loss—as many as six teeth; and (ii) in 2015, 24 children (ages 4-8) were infected with Nontuberculous Mycobacteria (NTM)—which can cause serious infections in the lungs, skin, lymph nodes or blood—which was traced to dental water at a particular dental practice in the State of Georgia.

Accordingly, there is an urgent need for improved equipment, devices, and/or methods for preventing, controlling, suppressing, restricting, and/or reducing bacteria and/or microorganism entry into dental units and associated waterlines. Moreover, there is an urgent need for eliminating, removing, and/or shocking of bacteria, biofilm, and/or other microorganisms that is present in dental units and/or DUWLs and/or for mitigating, reducing, removing, and/or eliminating bacteria and/or biofilm contamination from dental water reservoir systems (e.g., dental closed-bottle systems). There is an urgent need for improved equipment, devices, and/or methods for loading disinfectant (e.g., shock) solution into dental units and associated waterlines, as well as improved devices, equipment, and/or methods for holding and/or storing dental procedural/treatment water, as well as improved equipment, devices and/or methods for preventing bacteria and/or microorganism entry into dental units and associated waterlines, in order to improve safety for dental patients and staff as well as to increase the compliance rate amongst dental professionals to the CDC standard—which, as of the writing of this application—is only approximately 30%, given a current lack of equipment and methods for easy and cost-effective prevention of biofilm formation in dental units and waterlines and delivery of disinfectant (shock) solution into dental unit and associated waterlines. There is also a need to reduce exposure of antimicrobials, such as antibiotics, to the environment since microorganisms present in the environment can develop resistance to antimicrobials upon repeated exposure.

### SUMMARY

Provided herein are systems, kits and methods for disinfecting water lines that feed dental equipment.

Aspects of the disclosure are directed to a dental unit waterline systems (e.g., dental unit waterline purification systems) which include a water filter and a syringe. The water filter has an inlet and an outlet. The inlet of the water filter has a connector that is adapted to be releasably connected to a source dental waterline, and the outlet of the water filter has a connector that is adapted to be releasably connected the water inlet of a dental unit or associated waterline thereof. The syringe is suitable for containing disinfectant, sterilizing, or shock solution, and has a connector adapted to be releasably connected to the water inlet of a dental unit or associated waterline thereof.

Further aspects of the disclosure are directed to a method for disinfecting, sterilizing, or shocking a dental unit or associated waterlines, that includes (a) loading a syringe which includes a connector adapted to be releasably connected to the water inlet of a dental unit or associated waterline thereof, with disinfectant, sterilizing, or shock solution; (b) connecting the loaded syringe to the water inlet of the dental unit or associated waterline thereof, and (c) expressing the disinfectant, sterilizing, or shock solution contained within the syringe into the dental unit or associated waterline thereof.

Further aspects of the disclosure are directed to a dental liquid reservoir assembly which comprises (i) an upper portion having a connection or mating element for releasably attaching to a corresponding connection or mating element of a dental unit, and (ii) a lower base portion, wherein the upper portion and lower base portion have corresponding connection or mating elements so that the upper portion and lower base portion can be releasably connected to each other to form a reservoir assembly which can be filled with dental procedural liquids (e.g., water, medicaments, or “shock” solution) and be pressurized (e.g., up to 60 or 80 psi) such as when detachably connected to a dental unit (e.g., dental closed-bottle system) which provides pressurized air.

Further aspects of the disclosure are directed to a dental unit waterline system, as well as to methods for disinfecting or sterilizing using said system, which includes one or more water filters and a dental liquid reservoir assembly (“reservoir assembly”) (e.g., bottle) including an upper portion (e . . . g, a top portion) and a lower base portion (e.g., a bottom portion), wherein the upper and lower base portions are releasably connected to each other, such as to facilitate or enable cleaning, disinfecting or sterilizing of the internal surfaces of the liquid container. In aspects of the disclosure, the upper portion includes a threaded element for detachably connecting the upper portion to a mating threaded element on a dental unit, dental office or dental unit closed bottle system, e.g., dental chair water bottle system. In other aspects of the disclosure, one or more water filters of the dental unit waterline purification system is submersible within liquid (e.g., water) in the reservoir assembly and includes an input (or upstream) side which is adapted to receive liquid (e.g., water) flow into the filter and an output (or downstream) side which includes a connector adapted to be releasably connected (e.g., a Luer lock connector) to a mating connector on a down-straw of a dental unit, dental office or unit bottle system, or dental chair closed bottle system. In aspects of the disclosure, the dental unit is a dental chair and the dental unit waterline purification system is adapted to connect to a bottle system receiver of a dental office or dental chair which supplies treatment water and/or shock solution through all waterlines of a dental chair.

Further aspects of the disclosure are directed to a dental unit waterline system which includes a water filter having an input (or upstream) side which is adapted to receive water flow into the water filter and an output (or downstream) side which includes a connector adapted to be releasably connected (e.g., a Luer lock connector) to a mating connector on a down-straw of a dental unit, dental office or unit bottle system, or dental chair closed bottle system.

Still further aspects of the disclosure are directed to a disinfecting system and dental unit assembly including a first waterline, a second waterline, a third waterline, at least one sediment filter, a water filter, a dental unit, a dental handpiece, and a syringe. Each of the first, second, and third waterlines has a first end and a second end. The second end of the first waterline is adapted to be connected to a water source. Each of the at least one sediment filter has two quick disconnect fixtures. The water filter has an inlet and an outlet, and the inlet of the water filter is releasably connected to the second end of the first waterline. The outlet of the water filter is coupled to the second end of the second waterline. The dental unit has an inlet and an outlet, and the inlet of the dental unit is releasably connected to the second end of the second waterline. The outlet of the dental unit is releasably connected to the first end of the third waterline. The dental handpiece has an inlet releasably coupled to the second end of the third waterline. The syringe has a quick disconnect fitting adapted to be releasably connected to the inlets of the water filter and the dental unit.

In aspects of the disclosure, the syringe includes a disinfecting and/or sterilizing agent.

In aspects of the disclosure, the water filter prevents flow-through of particles, microorganisms, or the like which have an average particle size of 0.4 micrometers or more.

In aspects of the disclosure, the water filter prevents flow-through of particles, microorganisms, or the like which have an average particle size of 0.3 micrometers or more.

In aspects of the disclosure, the water filter prevents flow-through of particles, microorganisms, or the like which have an average particle size of 0.2 micrometers or more.

In aspects of the disclosure, the water filter prevents flow-through of particles, microorganisms, or the like which have an average particle size of 0.1 micrometers or more.

In aspects of the disclosure, the water filter is a microporous membrane filter.

Still further aspects of the disclosure are directed to a method for minimizing or preventing biofilm formation in dental unit that includes installing onto the source waterline to one or more dental units a water filter having an inlet and an outlet, the inlet of the water filter releasably connected to the source waterline, and the outlet of the water filter releasably connected to the dental unit or associated waterline thereof.

In aspects of the disclosure, the water filter prevents flow-through of particles, microorganisms, or the like which have an average particle size of 0.2 micrometers or more.

In aspects of the disclosure, the water filter prevents flow-through of particles, microorganisms, or the like which have an average particle size of 0.1 micrometers or more.

In aspects of the disclosure, the water filter is a microporous membrane filter.

In aspects of the disclosure, the water filter contains no antimicrobial agents.

In aspects of the disclosure, the water filter contains no oxidizing agents or metal ions.

In aspects of the disclosure, the water filter is a microporous membrane filter.

In aspects of the disclosure, the outlet of the water filter includes a Luer fitting and the first end of the second waterline includes a Luer fitting that is releasably connected to the Luer fitting of the second waterline.

In aspects of the disclosure, the dental unit is an ultrasonic scaler.

In aspects of the disclosure, the dental unit is an air-water syringe.

In aspects of the disclosure, the dental unit is a dental chair with its associated waterlines connected with treatment devices, such as, e.g., a high-speed handpiece, a low-speed handpiece, an air/water syringe, an ultrasonic scaler, and/or an air polisher.

In aspects of the disclosure, the disinfecting and/or sterilizing agent is selected from the group consisting of hypochlorites, glutaraldehyde, hydrogen, ortho-phthalaldehyde, peracetic or peroxyacetic acid, phenol derivatives, alcohol, and quaternary ammonium compounds.

Still further aspects of the disclosure are directed to methods for disinfecting and/or sterilizing a dental unit having a source or supply waterline to the dental unit, comprising connecting to a connection or mating element—which is present on the source waterline—a syringe which comprises a corresponding connection or mating element, and which is at least partially filled with a disinfectant and/or sterilizing agent; injecting the disinfectant and/or sterilizing agent into the dental system; permitting the disinfectant and/or sterilizing agent to reside in the dental unit and/or dental unit waterlines for a period sufficient to kill internal bacteria; and flush the disinfectant and/or sterilizing out of the dental unit and/or dental unit waterlines with bacteria-free water (such as water than has passed through a water filter having a sterilizing grade as described herein).

In aspects of the disclosure, the method includes injecting the disinfectant and/or sterilizing agent into the dental system until the disinfectant and/or sterilizing agent exits the dental handpiece.

In aspects of the disclosure, the method includes visually verifying that the disinfectant and/or sterilizing agent exits from the dental handpiece.

In aspects of the disclosure, the method includes providing the disinfectant and/or sterilizing agent with a dye to improve visualization of the disinfectant and/or sterilizing agent.

Further aspects of the disclosure are directed to a disinfecting and/or sterilizing kit for a dental system including a water filter, at least one sediment filter, a plurality of waterlines, a water filter, and a syringe. The water filter has an inlet and an outlet and quick disconnect fitting on the inlet and the outlet. Each waterline of the plurality of waterlines has a first end and a second end and quick disconnect fittings on the first end and the second end. The syringe has a quick disconnect fitting that is adapted to connect to the filter and to the plurality of waterlines.

In aspects of the disclosure, the dental unit waterline (purification) kit comprises a dental liquid reservoir assembly and optionally a water filter, e.g., having a sterilizing grade of 0.1 μm or 0.2 μm.

In aspects of the disclosure, the syringe includes a disinfecting and/or sterilizing agent.

In aspects of the disclosure, the disinfecting and/or sterilizing kit includes a dental handpiece.

In aspects of the disclosure, the dental unit is an ultrasonic scaler.

In aspects of the disclosure, the dental unit is a dental chair and its associated waterlines.

In aspects of the disclosure, the dental unit is a dental closed bottle system.

Further aspects of the disclosure are directed to a dental unit waterline kit (e.g., dental unit waterline kit, or dental unit waterline safety kit, or dental unit waterline compliance kit), such as for use with a dental closed-bottle system, which comprises a dental liquid reservoir assembly and a filter. In aspects of the disclosure, the dental liquid reservoir assembly (e.g., two component or multi-component dental liquid reservoir assembly, e.g., two component high-pressure dental reservoir or multi-component high-pressure dental liquid reservoir assembly) where the components include an upper portion and a separate lower base portion which are releasably attached to each other such as in a liquid tight manner, wherein the upper and lower base portions each comprise corresponding connection or mating elements. In aspects of the disclosure, the filter is adapted to be connected to the down-straw of the dental unit, the filter including a chemical free-filter member having a biopharmaceutical-grade microporous membrane to filter the water flow, the microporous membrane having a final sterilizing grade of at least 0.2 μm. In aspects of the disclosure, the filter is adapted to be connected to a down-straw of a dental unit and includes a chemical free-filter member having a biopharmaceutical-grade microporous membrane to filter the water flow. The microporous membrane has a final sterilizing grade of 0.1 μm or 0.2 μm.

In aspects of the disclosure, the filter member includes a hydrophilic membrane comprising polyethersulfone (PES) or similar filter membrane

In aspects of the disclosure, the filter member is formed from a material selected from the group consisting of PTFE, polypropylene, and polyethylene.

In aspects of the disclosure, the reservoir assembly (or dental liquid container) includes an upper portion and a lower base portion that are releasably coupled together, and the upper portion is separable from the lower base portion to provide access to an inner surface of the reservoir assembly for disinfection and/or sterilization as well as for ease of placement of a chemical straw, tablet, or water filter into the liquid reservoir (or water bottle).

In aspects of the disclosure, the filter is adapted to be releasably coupled to the down-straw of the dental unit.

In aspects of the disclosure, the filter includes a Luer connector for releasably coupling the filter to the down-straw of the dental unit.

In aspects of the disclosure, the water supply kit includes a sediment filter.

Further aspects of the disclosure are directed to a method of delivering purified water to a dental unit comprising removably connecting at least one component of a reservoir assembly (e.g., a multi component reservoir assembly) to the dental unit, removably connecting a water filter (e.g., a water filter rated to 0.2 μm or even 0.1 μm pass-through) to a draw-straw of the dental unit, and optionally delivering water from the reservoir assembly through the water filter and down-straw to the dental unit. In aspects of the disclosure, the method comprises pressuring the reservoir assembly, such as from the dental unit, e.g., to a internal psi of up to 50 psi (e.g., up to 60 psi, up to 70 psi, or even up to 80 psi). In aspects of the disclosure, the method comprises filling or partially filling the reservoir assembly with water and/or other liquid useful in dental procedures. In aspects of the disclosure, the method comprises connecting the lower base unit of the reservoir assembly to the upper portion following connection of the water filter to the down-straw.

In aspects of the disclosure, attaching the filter includes using Luer connectors to couple the filter to the down-straw.

### BRIEF DESCRIPTION OF THE DRAWINGS

A better understanding of the features and advantages of the disclosed technology will be obtained by reference to the following detailed description that sets forth illustrative aspects, in which the principles of the technology are utilized, and the accompanying figures of which:

FIG. 1 is a schematic representation of a dental system according to aspects of the disclosure;

FIG. 2 is an enlarged view of the indicated area of detail shown in FIG. 1 illustrating a connector;

FIG. 3 is a side view of an ultrasonic scaler insert, handpiece, and associated cabling including dental unit waterline, for use with the dental system shown in FIG. 1;

FIG. 4 is a schematic view of the dental system shown in FIG. 1 during a disinfecting and/or sterilizing procedure according to aspects of the disclosure;

FIG. 5 is a perspective, exploded view of a kit according to aspects of the disclosure;

FIG. 6 is a side view of the components of a kit according to other aspects of the disclosure;

FIG. 7 is a side view of the syringe of the kit components shown in FIG. 6 as the syringe is loaded with disinfecting and/or sterilizing agent;

FIG. 8 is a side view of the syringe shown in FIG. 7 with the disinfectant and/or sterilizing agent as it is removably attached to a dental unit waterline;

FIG. 9 is a side view of the syringe shown in FIG. 7 as the disinfectant and/or sterilizing agent is dispensed from the syringe into a waterline of the dental system;

FIG. 10 is a side view of the syringe and waterline shown in FIG. 9 after the disinfectant and/or sterilizing agent has been dispensed into the waterline of the dental system;

FIG. 11 is a side view of the first waterline, the water filter, and the second waterline of the dental system shown in FIG. 1 prior to connection of the components to each other;

FIG. 12 is a side view of the first waterline, the water filter, and the second waterline of the dental system shown in FIG. 1 after the components have been connected to each other;

FIG. 13 is a side view of the first waterline, the sediment filter, the water filter, and the second waterline of the dental system shown in FIG. 1 prior to connection of the first waterline, the sediment filter, and the water filter to each other with the second waterline connected to an outlet of the water filter;

FIG. 14 is a side cross-sectional view of a dental reservoir assembly according to aspects of the disclosure;

FIG. 15 is a side perspective exploded view of a dental reservoir assembly with down-straw of a dental unit (e.g., dental water bottle system) and a water filter releasably connected to the down-straw according to aspects of the disclosure;

FIG. 16 is a schematic view of a dental reservoir assembly releasably connected to a down-straw of a dental unit which is connected, or in fluid communication with, one or more downstream dental units according to aspects of the disclosure; and

FIG. 17 is a schematic view of a dental reservoir assembly releasably connected to a down-straw of a dental unit which is connected, or in fluid communication with, several downstream dental units or treatment devices according to aspects of the disclosure.

### DETAILED DESCRIPTION

Although illustrative systems of this disclosure will be described in terms of specific aspects, it will be readily apparent to those skilled in this art that various modifications, rearrangements, and substitutions may be made without departing from the spirit of this disclosure.

For purposes of promoting an understanding of the principles of this disclosure, reference will now be made to exemplary aspects illustrated in the figures, and specific language will be used to describe the same. It will nevertheless be understood that no limitation of the scope of this disclosure is thereby intended. Any alterations and further modifications of this disclosure features illustrated herein, and any additional applications of the principles of this disclosure as illustrated herein, which would occur to one skilled in the relevant art and having possession of this disclosure, are to be considered within the scope of this disclosure.

In the following description, well-known functions or constructions are not described in detail to avoid obscuring the disclosure in unnecessary detail.

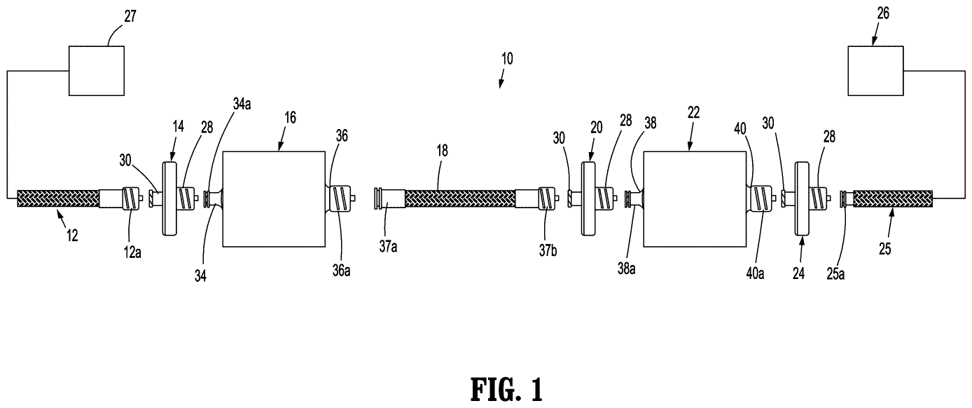

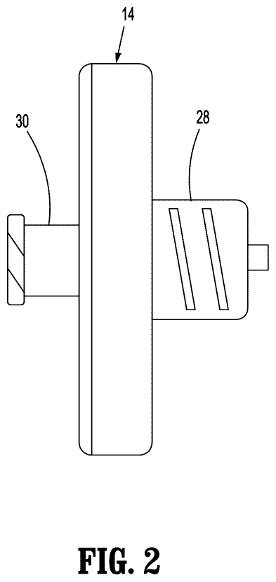

FIG. 1 illustrates a disinfection system for disinfecting and/or sterilizing water lines of dental units according to aspects of the disclosure shown generally as dental system 10. The dental system 10 includes a first waterline 12, a water filter 16, a second waterline 18, a dental unit 22, a third waterline 25, and a dental handpiece 26. The first waterline 12 is adapted to be coupled to a water source 27, e.g., a municipal water source or a self-contained water source, to supply water from the water source 27 through the filter 16 to the dental unit 22 and subsequently to the handpiece 26. In aspects of the disclosure, the dental system 10 can include one or more sediment filters, e.g., sediment filters 14, 20, 24, that remove particulate matter from the water supplied to the dental system 10 from the water source 10. Although three sediment filters are shown, it is envisioned that the dental system 10 may not include a sediment filter or can include any number of sediment filters, e.g., one or more. In aspects of the disclosure, the dental system 10 includes a single sediment filter 14 located between the water source 27 and the filter 16.

In aspects of the disclosure, the dental system 10 includes two or more (e.g., three or more, four or more, or even five or more) second waterlines that are downstream of the one or more water filters 16 and are associated with two or more (e.g., three or more, four or more, or even five or more) treatment devices or units 22 (e.g., high-speed handpiece, low-speed handpiece, air/water syringe, ultrasonic scaler, air polisher, or the like). In this and other aspects of the disclosure, elements 22-26 can collectively include a single dental treatment device or dental unit (e.g., dental chair, dental closed-bottle system, high-speed handpiece, low-speed handpiece, air/water syringe, ultrasonic scaler, air polisher, or the like).

In aspects of the disclosure each of the components of the system includes inlet and outlet ends that have quick connect couplings or fittings that function to couple the various components of the dental system 10 to each other in a fluid tight manner to facilitate fluid delivery from the water source 27 to the dental handpiece 26. The quick disconnect fittings can be in the form of any type of coupling or fitting that can secure the components to each other in a fluid tight manner. Such quick disconnect couplings or fittings include Luer slip connectors, Luer lock connectors, ENF it connectors, small bore connectors, barbed connectors, bayonet connectors, push-to-connect fittings, and camlock couplings. Although Luer lock connectors are illustrated in the figures, it is envisioned that any type of quick disconnect fitting can be used to connect the components of the dental system together.

In dental systems that include one or more sediment filters, e.g., sediment filters 14, 20, and 24, each of the sediment filters includes an inlet end having a first quick disconnect fitting and an outlet end having a second quick disconnect fitting. As illustrated, each of the sediment filters 14, 20, and 24 includes a male Luer fitting and a female Luer fitting. Each of the male and female Luer fittings is a standardized fitting which creates a secure, leak-free connection between components in a fluid transfer system. The male fitting 28 of a quick disconnect fitting is adapted to releasably engage a female fitting 30 of another quick disconnect fitting in a fast and efficient manner to releasably couple components together. Each of the sediment filters 14, 20, and 24 define a fluid pathway for fluid, e.g., water, to flow through the filter. Although the sediment filters 14, 20, and 24 are shown to have one male fitting 28 and one female fitting 30, it is envisioned that the sediment filters 14, 20, and 24 can have two female fittings 30 or two male fittings 28 suitable for connection to the components of the dental system 10 in use.

The Luer connectors can be slip Luer connectors or Luer lock connectors. Slip Luer connectors include a female fitting that defines a tapered or frustoconical bore and a male fitting that defines a frustoconical extension that is pressed into the frustoconical bore to join the female fitting to the male fitting. Luer lock connectors are like slip Luer connectors, but the male connector includes internal threading about the frustoconical extension that receives external threading on the female fitting to lock the male and female fittings to each other.

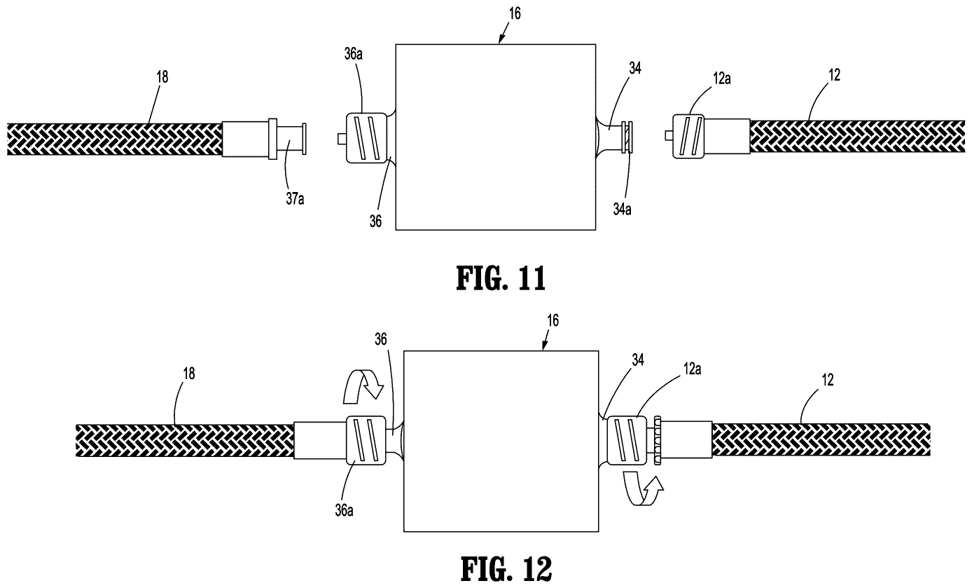

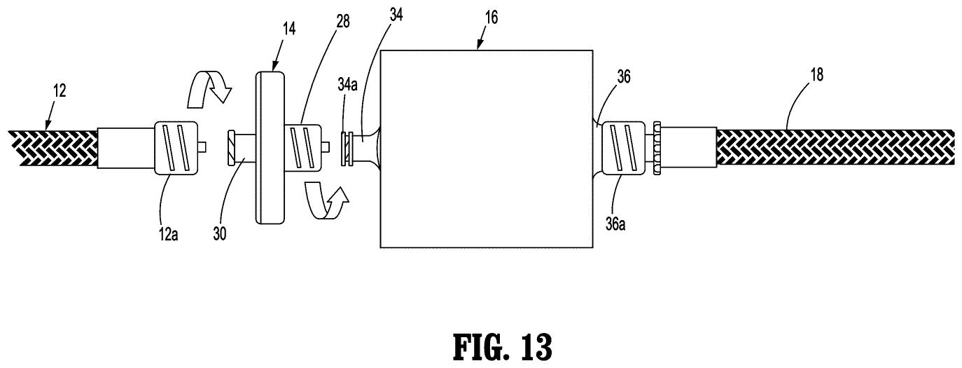

The first waterline 12 has a first end that is adapted to be coupled to the water source 27 and a second end that, in aspects of the disclosure, includes a quick disconnect fitting 12a (male or female) that can be releasably coupled to the sediment filter 14 (FIG. 13), or as shown in FIGS. 11 and 12 directly to the water filter 16. In aspects of the disclosure, the second end of the first waterline 12 includes a quick disconnect fitting 12a that is releasably coupled to a quick disconnect fitting 30 of the sediment filter 14.

The water filter 16 is provided to remove impurities, debris, and contaminants from the water flow upstream of the dental unit 22 and dental handpiece 26 to transform the water into ADA and CDC compliant water. In aspects of the disclosure, the water filter 16 has an inlet 34 and an outlet 36. The inlet 34 is connected to the sediment filter 14 to allow water exiting the sediment filter 14 to flow into the water filter 16 through the inlet 34 and exit the water filter 16 through the outlet 36. In aspects of the disclosure, the inlet 34 and the outlet 36 of the water filter 16 include quick disconnect fittings 34a and 36a (male or female) that facilitate releasable connection of the inlet 34 of the water filter 16 to the sediment filter 14 and releasable connection of the outlet 36 of the water filter 16 to the second waterline 18 to facilitate integration of the water filter 16 into any dental system 10 effortlessly and quickly.

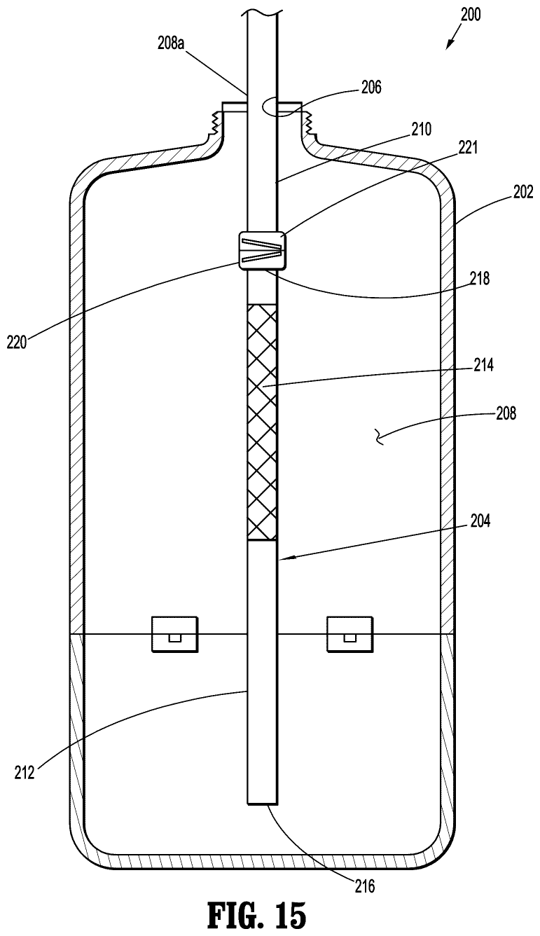

In aspects of the disclosure, such as is depicted in FIG. 15, the water filter 16 has an inlet 34 that lacks a quick disconnect fitting and which is adapted for receiving water, shock solution, or other liquids (such as any liquid that is present in a pressurized dental reservoir, liquid container, or bottle) and an outlet 36 which optionally includes a quick disconnect fitting, such as for releasably connecting to a down-straw of a dental bottle system (such as a down-straw associated with a conventional bottle system for a dental office or dental chair).

In aspects of the disclosure, the water filter 16 is a chemical free-water filter and may include a biopharmaceutical-grade microporous membrane to filter the water flow. Suitable liquid filter cartridges for in-line use are well-known and commercially available. For example, a cartridge utilizing a hydrophilic, polyethersulfone (PES) membrane, final sterilizing grade 0.1 μm or 0.2 μm can be suitable. Examples of other suitable membrane materials for providing a final sterilizing grade of 0.1 μm or 0.2 μm are PTFE, polypropylene and polyethylene. In aspects of the disclosure, the water filter 16 is positioned in close proximity to the dental unit to, in some cases, substantially reduce the length and/or amount of dental unit waterlines that are downstream of the filter and at risk of contamination and/or which require regular or periodic shock treatments of the dental unit and/or dental unit waterlines.

In aspects of the disclosure, the outlet 36 of the water filter 16 is coupled to the dental unit 22 or dental units 22 by the second waterline 18 and optional sediment filter 20. In aspects of the disclosure, the first end of the second waterline 18 includes a quick disconnect fixture 37a that is releasably connected to a quick disconnect fixture 36a formed on the outlet 36 of the water filter 16 and the second end of the second waterline 18 includes a quick disconnect fitting 37b that is connected to the inlet 38 of the dental unit 22 via the sediment filter 20. As described above, the sediment filter 20 includes male and female quick disconnect fittings 28 and 30 like the sediment filter 14 to facilitate effortless and quick coupling of the dental unit 22 to the second waterline 18 and to the dental handpiece 26. In aspects of the disclosure, the second waterline 18 releasably connects directly to the dental unit 22 without the presence of sediment filter 20.

In aspects of the disclosure, the filter is adapted to be releasably connected to a dental unit waterline and/or down-straw of the dental unit, the filter is chemical- and/or antibacterial- and/or antimicrobial-free filter, and the filter has a filtering grade of about 0.3 μm or less or even a filter grade of about 0.2 μm or less (e.g., a filtering grade between 0.5 μm and 0.3 μm, a filtering grade between 0.1 μm and 0.25 μm, a filtering grade between 0.1 μm and 0.25 μm, or a filtering grade between 0.5 μm and 0.2 μm).

In aspects of the disclosure, the dental unit 22 supplies electric, air, and/or water to one or more dental handpieces 26 (e.g., one or more downstream dental units or treatment devices or handpieces). Although the dental unit 22 is only shown to have a water inlet 38 and a water outlet 40, it is envisioned that the dental unit 22 may also include electrical and air inputs necessary to power the dental handpiece 26 coupled to the dental unit 22. As described above regarding the water filter 16, the inlet 38 and the outlet 40 of the dental unit 22 include quick disconnect fittings 38a and 40a (male or female) to facilitate effortless and quick coupling of the inlet 38 of the dental unit 22 to the sediment filter 20 (or directly to the second waterline 28) and of the outlet 40 of the dental unit 22 to the third waterline 25 and the dental handpiece 26. In that respect, the first end of the third waterline 25 includes a quick disconnect fitting 25a for connecting to the sediment filter 24 (or directly to the outlet 40 of the dental unit 22). In some aspects of the disclosure, a sediment filter is not provided between the dental unit 22 and the dental handpiece 26. Although not shown, a second end of the third waterline 25 can include a quick disconnect fixture for releasably coupling the third waterline 25 to the dental handpiece 26. In aspects of the disclosure, the third waterline 25 releasably connects directly to the handpiece 26 without the presence of sediment filter 24.

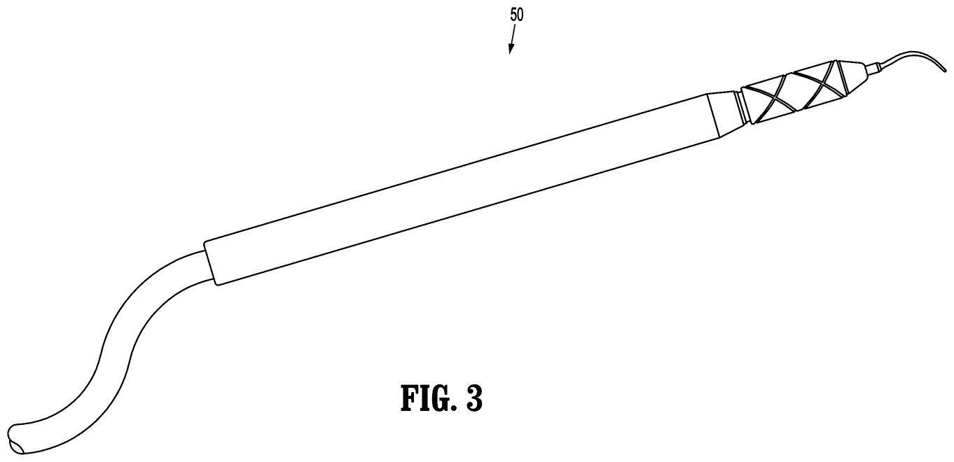

The dental handpiece 26 can be provided in a variety of types for performing a variety of different functions, e.g., scaling, polishing, suction, irrigation, etc. One type of dental handpiece 26 is an ultrasonic scaler 50 illustrated in FIG. 3. The ultrasonic scaler 50 is a dental instrument that is used for removing plaque, tartar, and biofilm from teeth using high-frequency vibrations and water irrigation. Ultrasonic scalers can be magnetostrictive scalers of piezoelectric scalers. Magnetostrictive scalers use magnetic fields to create vibrations in the ultrasonic scaler 50 whereas piezoelectric scalers use electrical currents delivered through piezoelectric crystals to generate vibrations in the ultrasonic scaler 50. In both types of ultrasonic scalers 50, water is delivered to the ultrasonic scaler 50 through a water channel in the ultrasonic scaler 50 to flush bacteria and debris from pockets in the gum between and around the teeth during a scaling procedure. As such, ultrasonic scalers 50 are susceptible to bacterial contamination. It is noted that the selected dental handpiece 26 must be coupled to a dental unit 22 suitable for powering and operating the dental handpiece 26. An example of a magnetostrictive ultrasonic scaler handpiece with associated cable (which includes an internal DUWL) is depicted in FIG. 3.

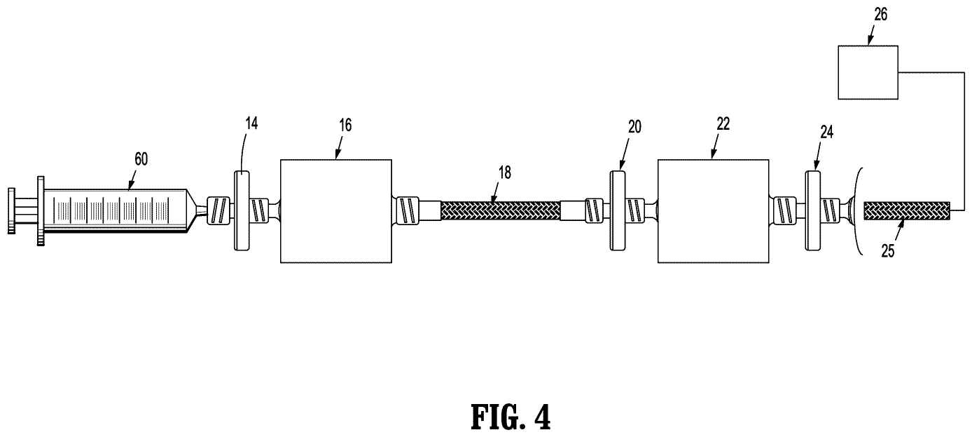

FIG. 4 illustrates the dental system 10 partially disassembled during a disinfecting and/or sterilizing procedure. To desterilize and/or disinfect the dental system 10 after one or more uses of the system, the sediment filter 14 (or the first waterline 12) is disconnected from the inlet 34 of the water filter 16 and a syringe 60 is used to inject a disinfecting and/or sterilizing agent into the fluid flow path of the remaining portion of the dental system 10. In aspects of the disclosure, the syringe 60 includes a body 62 defining a fluid reservoir 64, a plunger 66, and a fluid outlet 68 having the appropriate quick disconnect fitting 70. The quick disconnect fitting 70 is coupled to the inlet 34 of the water filter 16 and the plunger 66 is operated to inject the disinfectant and/or sterilizing agent through the water filter 16, the second waterline 18, the sediment filter 20, the dental unit 22, the third waterline 25, and the dental handpiece 26 to sterilize and/or disinfect the waterlines from the water filter 16 to the dental handpiece. Due to the close proximity of the water filter 16 to the dental handpiece, a minimal amount of disinfectant and/or sterilization agent is required to disinfect and/or sterilize the waterlines.

It is envisioned that the disinfecting and/or sterilization procedure described above can be performed at any point along the water flow path described above. For example, rather than disconnect the sediment filter 14 from the water filter 16 and injecting the disinfectant and/or sterilizing agent through the water filter 16 towards the dental handpiece 26, the sediment filter 20 can be disconnected from the dental unit 22 and the syringe 60 can be connected to the inlet 38 of the dental unit 22 to inject the disinfectant and/or sterilizing agent directly into the dental unit 22 and into the dental handpiece 26. In aspects of the disclosure, the syringe 60 can be connected to the second waterline 18 at quick disconnect fitting 37a, which, in the absence of sediment filter 20, connects directly to the inlet 38 of the dental unit 22. In the alternative, the sediment filter 24 can be disconnected from the third waterline 25 and the syringe 60 can be connected to the third waterline 25 to inject the disinfectant and/or sterilizing agent directly into the dental handpiece 26.

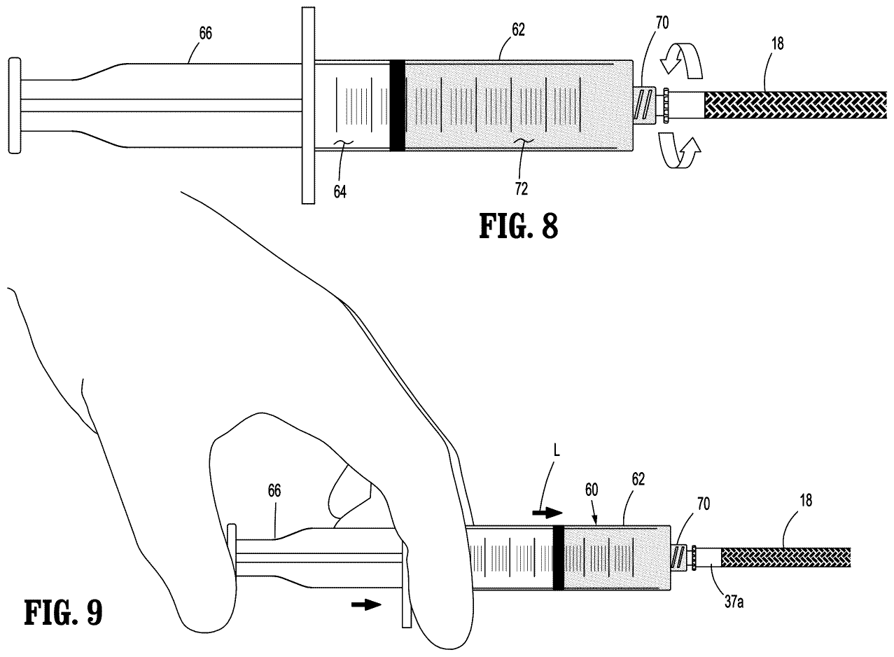

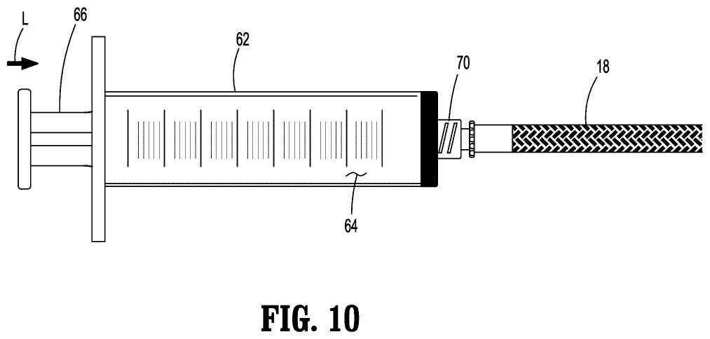

In aspects of the disclosure, the syringe 60 can be loaded with a disinfectant and/or sterilizing agent 72 by positioning the fluid outlet of the syringe 60 into a supply 73 (FIG. 7) of disinfecting agent 72 and withdrawing the plunger 66 in the direction indicated by arrow “K” in FIG. 7 to draw the disinfectant and/or sterilizing agent 72 from the supply 73 into the fluid reservoir 64 of the syringe 60. Once the syringe 60 is at least partially filled with the disinfecting and/or sterilizing agent 72 (FIG. 8), the quick disconnect fitting 70 of the syringe 60 can be coupled to the quick disconnect fitting of the second waterline 18, the dental unit 22, the third waterline 25, or any other component of the dental system 10. Once coupled, the plunger 66 of the syringe 60 can be moved in relation to the body 62 in the direction indicated by arrow “L” in FIGS. 9 and 10 to inject the disinfecting and/or sterilizing agent 72 into the remaining portion of the water flow path of the dental system 10 to disinfect and/or sterilize the components defining the water flow path.

Suitable disinfectant and/or sterilizing agents are known in the art and can be selected, e.g., from:
1. Hypochlorites which are available as liquid (e.g., sodium hypochlorite) or solid (e.g., calcium hypochlorite). The most prevalent chlorine products in the United States are aqueous solutions of 5.25%-6.15% sodium hypochlorite, usually called household bleach. Alternative compounds that release chlorine and are used in the health-care setting include demand-release chlorine dioxide, sodium dichloroisocyanurate, and chloramine-T.
2. Formaldehyde is sold and used principally as a water-based solution called formalin, which is 37% formaldehyde by weight. Aqueous formaldehyde solutions (1%-2%) also have been used to disinfect the internal fluid pathways of dialysis machines.
3. Glutaraldehyde is a saturated dialdehyde and a high-level disinfectant and chemical sterilant. Glutaraldehyde formulations include, e.g., ≥2% aqueous solutions of glutaraldehyde, buffered to pH 7.5-8.5 with sodium bicarbonate. Additional glutaraldehyde formulations are, e.g., glutaraldehyde-phenol-sodium phenate, potentiated acid glutaraldehyde, and stabilized alkaline glutaraldehyde).
4. Hydrogen peroxide solutions, e.g., 0.5% accelerated hydrogen peroxide; 3%, 7%, 10%, and 15% hydrogen peroxide. 7.5% hydrogen peroxide and 0.85% phosphoric acid.
5. Iodine solutions or tinctures. An iodophor is a combination of iodine and a solubilizing agent or carrier.
6. Ortho-phthalaldehyde is a high-level disinfectant that received FDA clearance in October 1999. It contains 0.55% 1,2-benzenedicarboxaldehyde (OPA).
7. Peracetic, or peroxyacetic, acid. Two chemical sterilants are available that contain peracetic acid plus hydrogen peroxide (i.e., 0.08% peracetic acid plus 1.0% hydrogen peroxide [no longer marketed]; and 0.23% peracetic acid plus 7.35% hydrogen peroxide.
8. Phenol derivatives commonly found as constituents of hospital disinfectants are ortho-phenylphenol and ortho-benzyl-para-chlorophenol. Other examples of phenolic disinfectant compounds are para-tertiary butylphenol, para-tertiary amylphenol and thymol.
9. Quaternary ammonium compounds such as benzalkonium chloride, alkyl dimethyl benzyl ammonium chloride, alkyl didecyl dimethyl ammonium chloride, and dialkyl dimethyl ammonium chloride. Newer quaternary ammonium compounds (i.e., fourth generation), are referred to as twin-chain or dialkyl quaternaries (e.g. didecyl dimethyl ammonium bromide and dioctyl dimethyl ammonium bromide).
10. Alcohols such as ethyl alcohol.

In aspects of the disclosure, the disinfectant and/or sterilizing agent can include a dye that allows for improved visualization of the disinfectant and/or sterilizing agent during a disinfecting and/or sterilizing procedure. More specifically, a dye that can be easily identified by a clinician can be included in the disinfectant and/or sterilizing agent when the disinfectant and/or sterilizing agent exit the dental handpiece during disinfecting and/or sterilizing procedure. This ensures that the disinfectant and/or sterilizing agent has passed through the entire flow path from the syringe 60 to the dental handpiece 26 during the disinfecting and/or sterilizing procedure. When a dye is used to improve visualization, the disinfecting and/or sterilization procedure also includes visually confirming that the disinfecting and/or sterilization has exited the dental handpiece 26.

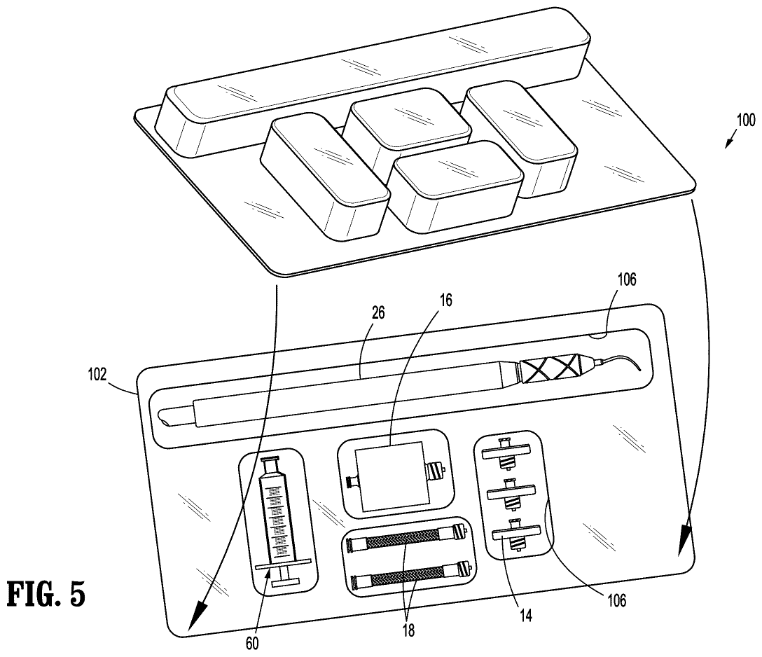

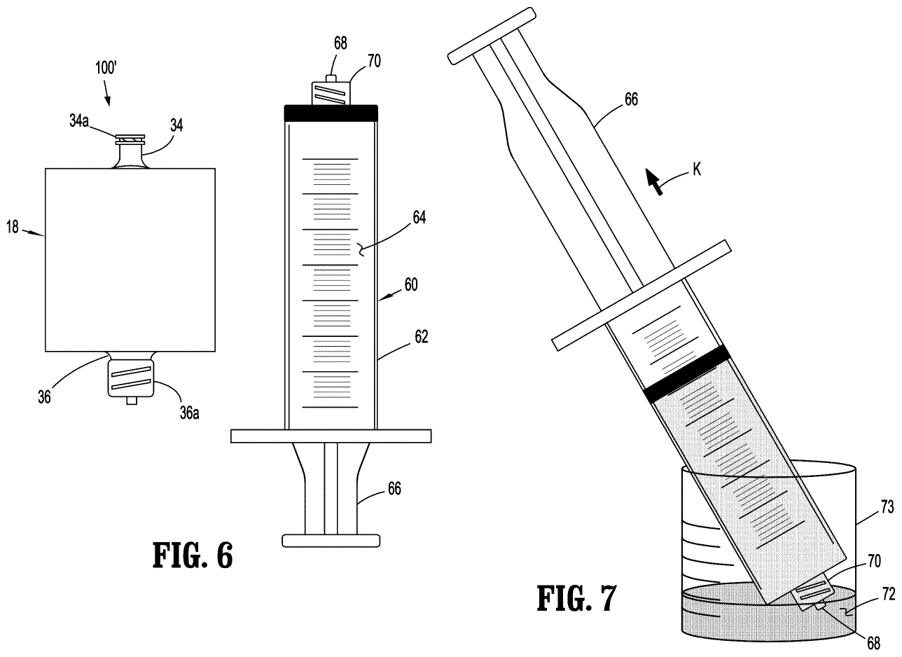

FIG. 5 illustrates a dental disinfecting and/or sterilization kit according to aspects of the disclosure shown generally as kit 100. The kit 100 includes a package 102 and one or more of the components of the dental system 10. For example, the kit can include one or more water filters 16, one or more sediment filters 14, one or more waterlines 18, one or more syringes 60, and a dental instrument 26. The syringe 60 can be preloaded with a disinfecting and/or sterilizing agent or empty to allow for filling later. The package 102, which may be a blister pack, includes recesses 106 to receive the components of the dental system 10 included in the kit 100 and can be hermetically sealed with a cover 108. In aspects of the disclosure, the kit 100′ (FIG. 6) includes only a syringe 60 and a water filter 18. In aspects of the disclosure, the syringe 60 can be included in the kit 100′ in an empty state and can be filled after removal from the kit 100, 100′.

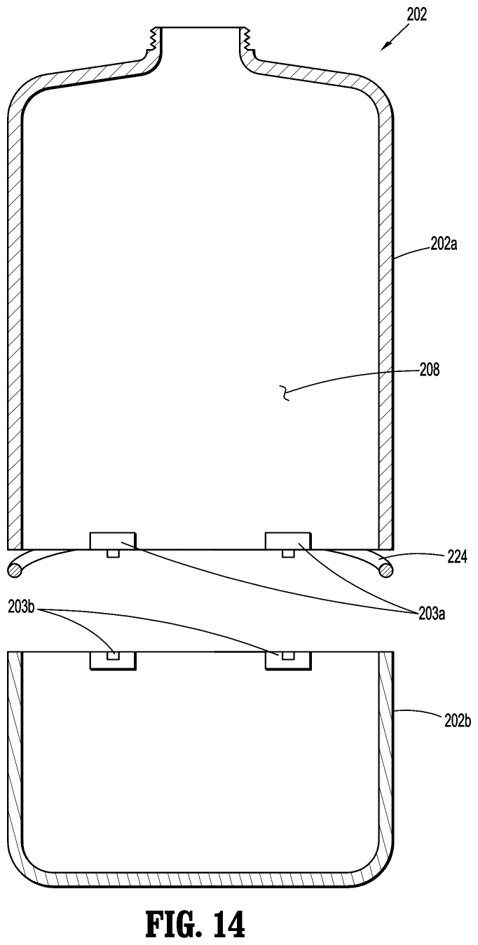

FIG. 14 illustrates a dental liquid reservoir assembly 202 (“reservoir assembly”) according to aspects of the disclosure for incorporation into the dental system 10 (FIG. 1). The reservoir assembly comprises an upper portion 202A and a lower base portion 202B that are releasably coupled together. In aspects of the disclosure, the upper portion 202A includes a first connector 203a (e.g., a threaded element) and the lower base portion 202B includes a second connector 203b (e.g., a mating threaded element). The first connector 203a and the second connector 203b can be releasably connected to each other to secure the upper portion 202A to the lower base portion 202B to define the cavity 208. In aspects of the disclosure, the first connector 203a and the second connector 203b can be disconnected or uncoupled to provide access to the inner portion of the reservoir assembly 202, such as, e.g., to facilitate disinfecting or sterilization of the inner walls of the reservoir assembly 202, to facilitate the filling of the reservoir assembly 202 with water, medicament and/or shock solution, to facilitate the placement of water treatment (e.g., silver- or iodine-based treatment) devices or water filters in the cavity 208 of the reservoir assembly, such as which would otherwise be too large to fit into a reservoir assembly not having first and second connectors, and/or to facilitate the shocking or disinfecting of dental unit(s) and associated waterlines by facilitating the loading of shock solution in to the water assembly. Although the interface between the upper portion 202A and the lower base portion 202B is shown to be centrally located on the reservoir assembly 202, it is envisioned that the interface could be at the upper end of the reservoir assembly 202 or the lower end of the reservoir assembly 202.

The first connector 203A and the second connector 203B are shown schematically and can be in the form of any type of connector capable of providing a fluid tight seal between the upper portion 202A and the lower base portion 202B. Examples of connectors include threads, friction fittings, clasps, or the like. It is envisioned that a seal or gasket 224, e.g. an O-ring, can be positioned and compressed between the upper portion 202A and the lower base portion 202B.

The reservoir assembly can be constructed of any suitable material or mixture there. Examples of suitable materials include, but are not limited to, polycarbonate, Polyethylene terephthalate glycol, HDPE (High-density polyethylene), stainless steel, or the like.

In aspects of the disclosure, the water filter 204 (FIG. 15) has a tubular body 212 that contains the filter member, e.g., the microporous membrane 214, and includes a water inlet 216 and a water outlet 218 with the microporous membrane 214 positioned between the water inlet 216 and the water outlet 218. In aspects of the disclosure, the tubular body 212 includes a connector 220, e.g., a Luer connector, that is positioned about the water outlet 218 and is adapted to be releasably connected to a connector 221 that is positioned about an inlet end 222 of the down-straw 210 of the dental unit 22 (FIG. 4). Alternatively, the water filter 204 can have a variety of configurations that include a water inlet and a water outlet and a microporous membrane positioned there between. In aspects of the disclosure, the water filter is connected to the downstream in such a manner that approximately one quarter inch of clearance remains at the bottom of the reservoir assembly.

FIG. 15 illustrates a reservoir assembly 202 according to aspects of the disclosure, with water filter 204 situated internally. The reservoir assembly 202 defines an opening 206 and a cavity 208 that communicates with the opening 206 for receiving water or other fluid (e.g., medicant or shock solution). The upper portion 202A of the reservoir assembly 202 comprises a connection or mating element (e.g., a threaded portion) (illustrated in the figure at the top of 202A) proximate to the top of the upper portion which is adapted to releasably attach to a connection or mating element (e.g., a corresponding threaded receptable) (not illustrated in the figure) of a dental unit, whereby the opening 206 forms a liquid and/or air (pressure) tight connection or seal with the connection element of the dental unit (e.g., in such a manner as to permit pressurization of the reservoir assembly by the dental unit, e.g., up to 50 psi, up to 60 psi, up to 70 psi, or even up to 80 psi) such that no liquid and/or air leakage or escape occurs from the now connected or mated reservoir assembly (with dental unit).

The water filter 204 includes a filter member 214 that is like the water filter 16 (FIG. 1). In aspects of the disclosure, the filter member 214 is chemical-, antibiotic-, and/or antimicrobial-free (“chemical free-”) filter member, e.g., which includes a biopharmaceutical-grade microporous membrane to filter the water flow. Utilization of chemical-free filters that prevent microorganisms from entering the waterlines carry several benefits. Moreover, the chemical-free or antibiotic- and/or antimicrobial-free water filter advantageously provides a mechanism for reducing use of antimicrobials such as antibiotics in dental unit systems as compared to commercially available dental waterline devices (e.g., chemical straws or tablets) which release and exposure both large numbers of patients and dental professionals, on a daily basis, to antibacterial and/or antimicrobial agents or the like. In this manner, environmental exposure to antimicrobials can be reduced in an effort to prevent or reduce development of microorganism resistance to antimicrobials. It should be understood that the terms “antimicrobials” or “antimicrobial” are meant to have their ordinary meaning and include, e.g., antibacterials, antivirals, antifungals and antiparasitics. Suitable water filter members for in-line use are well-known and commercially available. For example, a filter member 214 utilizing a hydrophilic, polyethersulfone (PES) membrane, final sterilizing grade 0.1 μm or 0.2 μm can be suitable. Examples of other suitable membrane materials for providing a final sterilizing grade of 0.1 μm or 0.2 μm are PTFE, polypropylene and polyethylene. In aspects of the disclosure, the filter member 114 is positioned in close proximity to the dental unit 22 and the dental handpiece 26 to reduce the length and/or amount of office waterlines (downstream of the filter) which require regular shock treatments of the dental system 10 consistent with CDC recommendations.

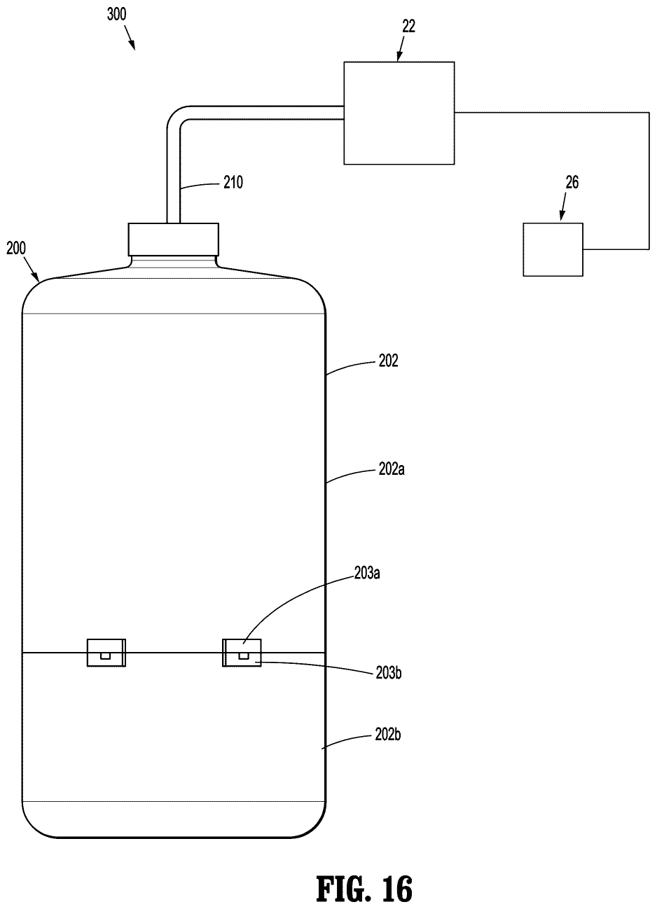

FIG. 16 illustrates a schematic view of a dental system 300 including the reservoir assembly 200. In aspects of the disclosure, the down-straw 210 of the dental unit 22 is connected to the water filter 204 by connecting the connector 220 (FIG. 14) of the water filter 204 to the connector 221 (FIG. 15) of the down-straw 210 of the dental unit 22. The outlet to the dental unit 22 is coupled to any number of downstream dental units, dental treatment devices, and/or dental treatment handpiece(s) 26, e.g., a high-speed handpiece, a low-speed handpiece, an air/water syringe, an ultrasonic scaler, an air polisher, or the like. In aspects of the disclosure, a sediment filter 24 can be interposed between down-straw 210 and the dental unit 22.

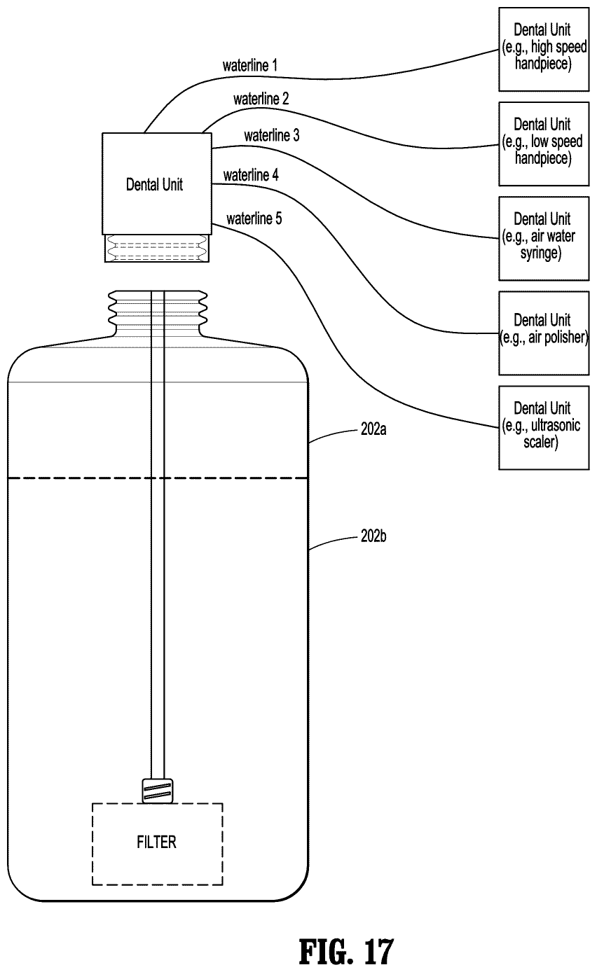

FIG. 17 illustrates schematic view of a dental system that includes a two-component reservoir assembly and water filter, operably connected to a dental unit that is coupled to several (i.e., five) downstream dental units in aspects of this disclosure.

In aspects of the disclosure, the reservoir assembly 202 can be filled or partially filled, with disinfectant, sterilizing, or shock solution to deliver the disinfectant, sterilizing, or shock solution to the dental unit 22 and to the dental handpiece 26 to sterilize the water lines of the dental unit 22 and the dental handpiece 26. The disinfectant, sterilizing, or shock solution within the reservoir assembly 202 will also disinfect or sterilize the inner walls of the reservoir assembly 202.

Further aspects of the disclosure are provided by the subject matter of the following clauses.

A dental unit waterline purification kit comprising: a water filter having an inlet, an outlet, and a filter member, the inlet of the water filter having a first connector adapted to be releasably connected to a first dental waterline, and the outlet of the water filter having a second connector adapted to be releasably connected to a second dental waterline; and a syringe suitable for containing dental waterline disinfectant, sterilizing, or shock solution, and having a third connector adapted to be releasably connected to a water inlet of a dental unit or associated waterline thereof.

The dental unit waterline purification kit according to any preceding clause, wherein the syringe includes a disinfecting and/or sterilizing agent.

The dental unit waterline purification kit according to any preceding clause, wherein the filter member of the water filter is adapted to prevent flow-through of particles, microorganisms, or the like which have an average particle size of 0.4 micrometers or more.

The dental unit waterline purification kit according to any preceding clause, wherein the filter member of the water filter is adapted to prevent flow-through of particles, microorganisms, or the like which have an average particle size of 0.1 micrometers or more.

The dental unit waterline purification kit according to any preceding clause, wherein the filter member of the water filter is a microporous membrane filter.

The dental unit waterline purification kit according to any preceding clause, wherein the water filter contains no antimicrobial or antibacterial agents.

The dental unit waterline purification kit according to any preceding clause, wherein the first, second and third connectors are Luer fittings.

The dental unit waterline purification kit according to any preceding clause, wherein the disinfecting and/or sterilizing agent is selected from the group consisting of hypochlorites, glutaraldehyde, hydrogen, ortho-phthalaldehyde, peracetic or peroxyacetic acid, phenol derivatives, alcohol, and quaternary ammonium compounds.

The dental unit waterline purification kit according to any preceding clause, wherein the filter member includes a hydrophilic, polyethersulfone (PES) membrane.

A water filter for a dental unit waterline comprising: an inlet, an outlet, and a filter member, the outlet of the water filter having a connector adapted to be releasably connected the water inlet of a dental unit or associated waterline thereof, the filter member adapted to prevent flow-through of particles, microorganisms, or the like which have an average particle size of 0.4 micrometers or more.

The water filter according to any preceding clause, wherein the filter member of the water filter is adapted to prevent flow-through of particles, microorganisms, or the like which have an average particle size of 0.3 micrometers or more.

The water filter according to any preceding clause, wherein the filter member of the water filter is adapted to prevent flow-through of particles, microorganisms, or the like which have an average particle size of 0.2 micrometers or more.

The water filter according to any preceding clause, wherein the filter member of the water filter is adapted to prevent flow-through of particles, microorganisms, or the like which have an average particle size of 0.1 micrometers or more.

The water filter according to any preceding clause, wherein the filter member of the water filter is a microporous membrane filter.

The water filter according to any preceding clause, wherein the water filter contains no antimicrobial or antibacterial agents.

The water filter according to any preceding clause, wherein the connector is a Luer fitting.

The water filter according to any preceding clause, wherein the filter member includes a hydrophilic, polyethersulfone (PES) membrane.

A dental unit waterline kit comprising: a dental water reservoir assembly comprising an upper portion and a lower base portion that are releasably connectable to each other in a liquid and air tight manner, wherein the upper and lower base portions each include corresponding connection or mating elements; and a filter adapted to be releasably connected to a down-straw of the dental unit, the filter being a chemical- and/or antibacterial- and/or antimicrobial-free filter and having a filtering grade between 0.5 μm and 0.25 μm.

The dental unit waterline kit according to any preceding clause, wherein the filter member includes a hydrophilic, polyethersulfone (PES) membrane.

The dental unit waterline kit according to any preceding clause, further comprising a sediment filter.

Persons skilled in the art will understand that the structures and methods specifically described herein and illustrated in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effectuated by one skilled in the art without departing from the scope or spirit of the disclosure. Additionally, it is envisioned that the elements and features illustrated or described in connection with one exemplary aspect may be combined with the elements and features of another without departing from the scope of this disclosure, and that such modifications and variations are also intended to be included within the scope of this disclosure. Indeed, any combination of any of the disclosed elements and features is within the scope of this disclosure. Accordingly, the subject matter of this disclosure is not to be limited by what has been particularly shown and described.

1. A dental unit waterline purification kit comprising:
a. a water filter having an inlet, an outlet, and a filter member, the inlet of the water filter having a first connector adapted to be releasably connected to a first dental waterline, and the outlet of the water filter having a second connector adapted to be releasably connected to a second dental waterline; and
b. a syringe suitable for containing dental waterline disinfectant, sterilizing, or shock solution, and having a third connector adapted to be releasably connected to a water inlet of a dental unit or associated waterline thereof.

2. The dental unit waterline purification kit of claim 1, wherein the syringe includes a disinfecting and/or sterilizing agent.

3. The dental unit waterline purification kit of claim 1, wherein the filter member of the water filter is adapted to prevent flow-through of particles, microorganisms, or the like which have an average particle size of 0.4 micrometers or more.

4. The dental unit waterline purification kit of claim 1, wherein the filter member of the water filter is adapted to prevent flow-through of particles, microorganisms, or the like which have an average particle size of 0.1 micrometers or more.

5. The dental unit waterline purification kit of claim 1, wherein the filter member of the water filter is a microporous membrane filter.

6. The dental unit waterline purification kit of claim 1, wherein the water filter contains no antimicrobial or antibacterial agents.

7. The dental unit waterline purification kit of claim 1, wherein the first, second and third connectors are Luer fittings.

8. The dental unit waterline purification kit of claim 2, wherein the disinfecting and/or sterilizing agent is selected from the group consisting of hypochlorites, glutaraldehyde, hydrogen, ortho-phthalaldehyde, peracetic or peroxyacetic acid, phenol derivatives, alcohol, and quaternary ammonium compounds.

9. The dental unit waterline purification kit of claim 1, wherein the filter member includes a hydrophilic, polyethersulfone (PES) membrane.

10. A water filter for a dental unit waterline comprising:
an inlet, an outlet, and a filter member, the outlet of the water filter having a connector adapted to be releasably connected the water inlet of a dental unit or associated waterline thereof, the filter member adapted to prevent flow-through of particles, microorganisms, or the like which have an average particle size of 0.4 micrometers or more.

11. The water filter of claim 10, wherein the filter member of the water filter is adapted to prevent flow-through of particles, microorganisms, or the like which have an average particle size of 0.3 micrometers or more.

12. The water filter of claim 11, wherein the filter member of the water filter is adapted to prevent flow-through of particles, microorganisms, or the like which have an average particle size of 0.2 micrometers or more.

13. The water filter of claim 12, wherein the filter member of the water filter is adapted to prevent flow-through of particles, microorganisms, or the like which have an average particle size of 0.1 micrometers or more.

14. The water filter of claim 10, wherein the filter member of the water filter is a microporous membrane filter.

15. The water filter of claim 10, wherein the water filter contains no antimicrobial or antibacterial agents.

16. The water filter of claim 10, wherein the connector is a Luer fitting.

17. The dental unit waterline purification kit of claim 10, wherein the filter member includes a hydrophilic, polyethersulfone (PES) membrane.

18. A dental unit waterline kit comprising:
a dental water reservoir assembly comprising an upper portion and a lower base portion that are releasably connectable to each other in a liquid and air tight manner, wherein the upper and lower base portions each include corresponding connection or mating elements; and a filter adapted to be releasably connected to a down-straw of the dental unit, the filter being a chemical- and/or antibacterial- and/or antimicrobial-free filter and having a filtering grade between 0.5 μm and 0.25 μm.

19. The dental unit waterline kit of claim 18, wherein the filter member includes a hydrophilic, polyethersulfone (PES) membrane.

20. The dental unit waterline kit of claim 18, further comprising a sediment filter.
